(19) United States
(12) Patent Application Publication
(10) Pub. No.: US 2010/0082294 A1
(43) Pub. Date: Apr. 1, 2010

(54) COMMISSIONING AND USER SYSTEM FOR RADIATION THERAPY TREATMENT DEVICES

(75) Inventor: Nabil Adnani, Wexford, PA (US)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: D3 Radiation Planning, LP, Pittsburgh, PA (US)

(21) Appl. No.: 12/570,970

(22) Filed: Sep. 30, 2009

Related U.S. Application Data
(60) Provisional application No. 61/136,771, filed on Oct. 1, 2008.

(51) Int. Cl. G21C 17/00; A61N 5/10

(52) U.S. Cl. 702/182; 378/65

(57) ABSTRACT

This invention is generally directed to a method, system and computer program product for commissioning an RTD. The method comprises entering identification data and machine information for an RTD into a database, importing scan data measuring beam characteristics, entering additional data from measurement results generated by test equipment measuring aspects of the RTD's beam into the RTD database, creating a Data Book in the database comprising output factors, transmission factors and processed scan data; comparing information in the Data Book with information according to a predefined standard for commissioning the RTD, comparing a treatment plan MU based on a set of treatment parameters to the RTD MU calculated using the set of treatment parameters, and commissioning the RTD based at least in part on if the comparing meets the predefined standard and if the MU calculated by the RTD is within an acceptable range of the treatment plan MU.

### CROSS-REFERENCE TO RELATED PATENT APPLICATIONS

This patent application claims the benefit of U.S. Provisional Patent Application No. 61/136,771, filed Oct. 1, 2008.

### FIELD OF THE INVENTION

This invention generally pertains to commissioning systems and user support systems for radiation therapy treatment devices (“Radiation Treatment Devices.”)

### BACKGROUND

The commissioning of a Radiation Treatment Device (RTD) that emits a radiation beam requires the acquisition and processing of a large amount of physics data. Such commissioning verifies that the RTD is safe to be used for radiation delivery for treatment purposes. It also allows for the beam characteristics of the RTD to be used by treatment planning systems in conjunction with 3D patient images to calculate the distribution of a radiation dose in and around the diseased target as a function of the beam on-time (commonly referred to as Monitor Units). If the process is not done correctly, the commissioning of the RTD and its use on a patient may be delayed or may possibly result in serious consequences to a patient' treatment outcome and safety.

Currently to commission an RTD, a checklist of all required data to generate the beam models for the treatment planning system is prepared. Any data needed for other calculations not required by the planning system is added to the checklist. The commissioning physicist uses sophisticated equipment to scan the RTD's radiation beam to determine all of its characteristics, processes the scanned data and exports it in a format that the planning system can use. Any measurements that are not provided by the scanning equipment are also taken and recorded. A book is generated from the scanned data and the other recorded measurements. Copies of the book are printed and distributed to appropriate locations within the clinic where the RTD is to be used.

A need exists for a rigorous process to commission an RTD that minimizes errors and provides a comprehensive collection of data (a “Data Book”) associated with the RTD. A need further exists for a tool to manage the administrative aspects of an RTD. A need also exists for a method and tool to quickly query the radiation beam parameters needed to generate the dose to be delivered to a patient in a clinical setting and to perform verifications of the planning system's calculations.

### SUMMARY

This invention is generally directed to providing a method and a computer program product adapted to be executed to implement such a method for commissioning a radiation treatment device (RTD). The method comprises entering identification data for an uncommissioned RTD into an RTD database; entering machine information for the RTD into the RTD database; importing into the RTD database processed scan data generated by scanning equipment measuring beam characteristics of the RTD's beam; entering additional data from measurement results generated by test equipment measuring aspects of the RTD's beam into the RTD database; creating a Data Book in the RTD database comprising output factors, transmission factors and processed scan data; comparing information in the Data Book with information according to a predefined standard for commissioning the RTD; comparing a treatment plan MU based on a set of treatment parameters to the RTD MU calculated using the set of treatment parameters; and commissioning the RTD. The decision to commission the RTD is based, at least in part, on whether the comparing meets the predefined standard and whether the MU calculated by the RTD is within an acceptable range of the treatment plan MU.

In another embodiment there is a system for commissioning a radiation treatment device (RTD) comprising: a server including a processor and a data store including an RTD database, the server accessed by the user interface, the server retrieving data related to the commissioning of the RTD; an user interface comprising an input and an output; first test equipment that has an output providing processed scan data obtained from the RTD; second test equipment that has an output providing additional data from the RTD; a first algorithm deriving a Data Book residing in the RTD database comprising output factors, transmission factors and processed scan data; data representing a predefined standard for commissioning the RTD; a second algorithm for comparing information in the Data Book with information according to a predefined standard for commissioning the RTD; and a third algorithm for comparing a treatment plan MU based on a set of treatment parameters to the RTD MU calculated using the set of treatment parameters.

As defined herein, the term “Data Book” shall mean an electronic or hard copy collection of data necessary for the dose calculations used with that RTD when treating patients. This collection of data may include any or all of the following, but is not limited to: PDDs, TMRs, OARs, OFs, and TFs necessary for the dose calculations used with that RTD when treating patients. The data in the Data Book are used as inputs into the specific formulas that calculate the beam on-time needed to deliver a particular dose in a given treatment scenario. It may also be used to independently verify the MUs calculated by the treatment planning system or similar software.

The following definitions and acronyms are used herein:
Data Book an electronic or hard copy collection of data necessary for the dose calculations used with an RTD when treating patients
MLC Multi Leaf Collimator
MU monitor unit—the unit of beam on-time for an RTD
OAR Off Axis Ratio—the dose at a distance from the central axis expressed as a percentage of the dose at the isocenter
OF Output Factor—the ratio between the measured radiation dose at a reference point in a given geometry to that for a reference geometry
PDD Percent Depth Dose—the dose expressed as a percentage of the maximum dose at depth
RTD Radiation Treatment Device—any type of radiation therapy treatment device, including but not limited to, linear accelerators
SRS Stereotactic Radio Surgery
TF Transmission Factor
TMR Tissue Maximum Ratio—the ratio of the dose at a given point to the dose at depth of maximum dose or dₘₐₓ (both points are at isocenter distance from the radiation source)

### BRIEF DESCRIPTION OF THE DRAWINGS

The above-noted and other advantages of the invention will be apparent from the description of the invention provided herein with reference to the attached drawings in which:

FIG. 1 illustrates an embodiment of a system for commissioning an RTD;

FIG. 2 illustrates another embodiment of a system for commissioning an RTD;

FIG. 3 is a flow chart of the method according to an embodiment of the invention;

FIG. 4 an embodiment of a screen for inputting the technical details for a RTD;

FIG. 5 illustrates one embodiment of a screen for entering OFs into the user interface;

FIG. 6 illustrates one embodiment of a screen for comparing beam data to reference data;

FIG. 7 illustrates one embodiment of a screen for querying beam data;

FIG. 8 illustrates one embodiment of a screen for managing machine related documents;

FIG. 9 illustrates one embodiment of a Data Collection Checklist;

FIG. 10 illustrates one embodiment of a Monthly Calibration Screen; and

FIG. 11 illustrates one embodiment of patient's electronic chart accessible through the user interface.

### DETAILED DESCRIPTION OF THE EMBODIMENTS

The embodiment of the invention described below is not intended to be exhaustive or to limit the invention to the precise structure and operation disclosed. Rather, the embodiment described in detail below has been chosen and described to explain the principles of the invention and its application, operation and use in order to best enable others skilled in the art to follow its teachings.

This invention is generally directed to a method, system and computer program product for commissioning RTDs and providing a support system for clinical staff using such commissioned RTDs. The following examples will use the commissioning of linear accelerator and the subsequent support system for staff using such RTD to illustrate the application of the invention but such use of a particular type of RTD should not be construed as in any way as limiting the scope of the invention.

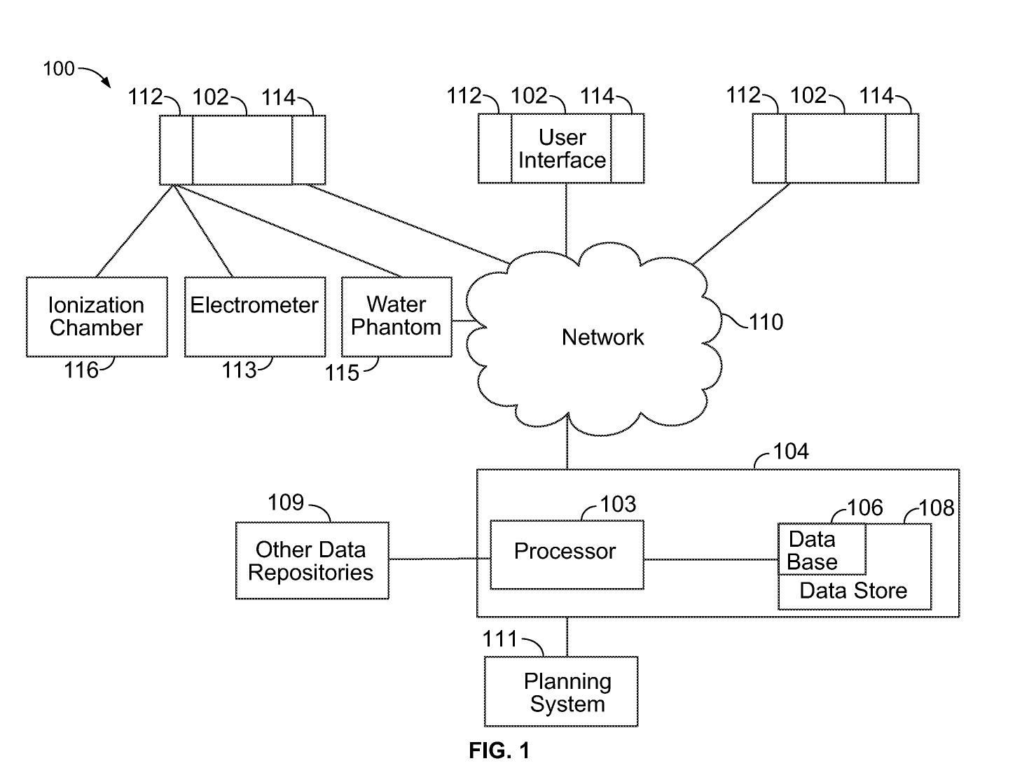

Turning now to FIG. 1, one embodiment of a system for commissioning a RTD is shown. The system 100 includes at least one user interface 102, a server 104 and a network 110. The server 104 may include a processor 103 and a RTD database 106 in a non volatile memory data store 108. The user interface 102 includes a user input 112 and a user output 114. The user interface 102 may be a personal computer, a laptop or any other appropriate interface. The user interface may connect through a network 110 to the server 104 or may connect directly to the server 104. The network 110 may be a Local Area Network or a Wide Area Network. The input 112 to the user interface 102 may be a touch screen device, a mouse, keyboard or any other appropriate input device for a user interface. The output 114 to the user interface 102 may be a LCD display with a touch screen, printer or any other appropriate device display capable of displaying information to a user. Other data repositories 109 may be connected to the server 104. Other applications such as a radiation planning system 111 may be connected to the server 104 directly or through the network 110. Test equipment, for example an electrometer 113 and an ionization chamber 116, may be connected to the user interface 102 to input data directly to the user interface 102. Test equipment, such as a water phantom 115, may be connected to user interface 102 to make test data, such as raw and processed scan data, available to be imported into the user interface 102.

In operation, the server 104 is accessed by the user interface 102 to facilitate the transfer of information from one user interface 102 to another. The server 104 may be accessed by the user interface 102 to run a display application program on the user interface 102 and to retrieve data stored in the database 106. In the preferred embodiment, a designated administrator of the system 100 creates user accounts (usernames and passwords) with specific access privileges. These privileges may also include authorization privileges for approving documents stored in the database 106. This is used by the administrator of the system 100 to add, remove and determine user access rights and privileges. When a particular feature is not accessible to a user, it may be deactivated for that user.

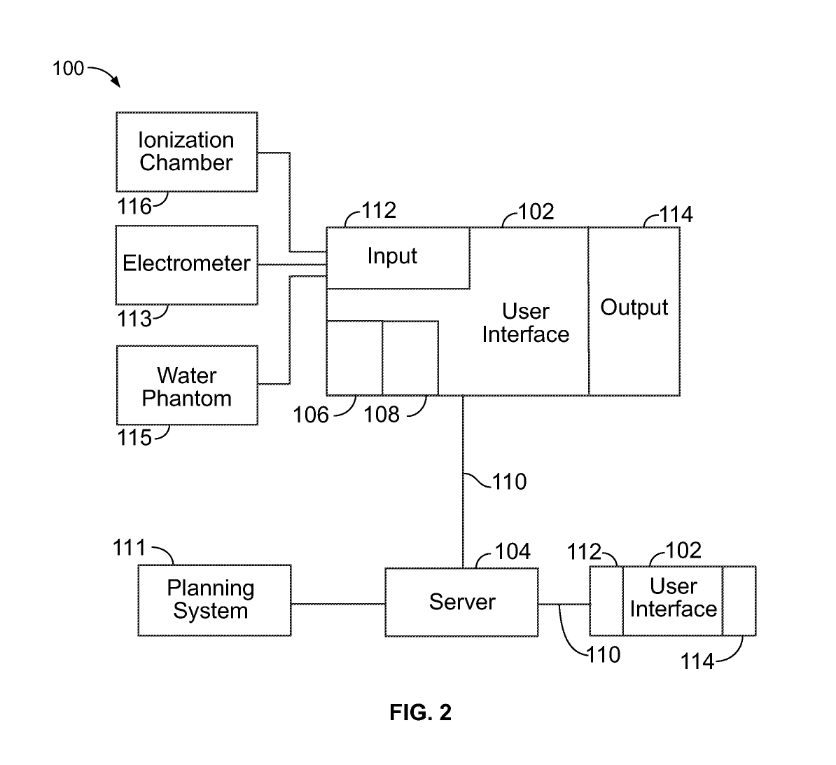

FIG. 2 illustrates another embodiment of a system 100 for commissioning an RTD according to the invention. Turning now to FIG. 2, the system 100 includes at least one user interface 102, a server 104, and a database 106 in non volatile memory 108. The user interface 102 includes a user input 112 and a user output 114. The user interface 102 may be a personal computer, a laptop or any other appropriate interface. The user interface(s) 102 may connect directly to the server 104. The database 106 and non volatile memory 108 may be part of the user interface 102. The user interface 102 connects directly to the database 106 in the non-volatile memory data store 108. The input 112 to the user interface 102 may be a touch screen device, a mouse, keyboard or any other appropriate input device for a user interface 102. The output 114 to the user interface 102 may be a LCD display with a touch screen, printer or any other appropriate device display capable of displaying information to a user. Test equipment, for example an electrometer 113, and an ionization chamber 116 to measure output and transmission factors (for instance) may be connected to the user interface 102 to input data directly to the user interface 102. Test equipment, such as a water phantom 115, may be connected to the user interface to make test data, such as raw and processed scan data, available to be imported into the user interface 102. Other applications such as a radiation planning system 111 may be connected to the server 104.

In operation according to the embodiment illustrated in FIG. 2, the server 104 is accessed by the user interface 102 to facilitate the transfer of information from one user interface to another. In the preferred embodiment, the server 104 is a File Transfer Protocol (FTP) server used for the transfer of information from one user interface 102 to another 102. The centralized FTP server allows for the commissioning of an RTD to be done at a remote site. Data entered in the user interface 102 at a remote site is synchronized with data contained in the database 106 so that only data that has been changed is updated. A list of designated recipients associated with an RTD may, in some embodiments, receive messages (e-mail or otherwise) notifying them of changes to the RTD's data in the database 106. A designated administrator of the system 100 creates user accounts (usernames and passwords) with specific access privileges. This is used by the administrator of the system 100 to add, remove and determine user access rights and privileges. When a particular feature is not accessible to a user, it may be deactivated for that user.

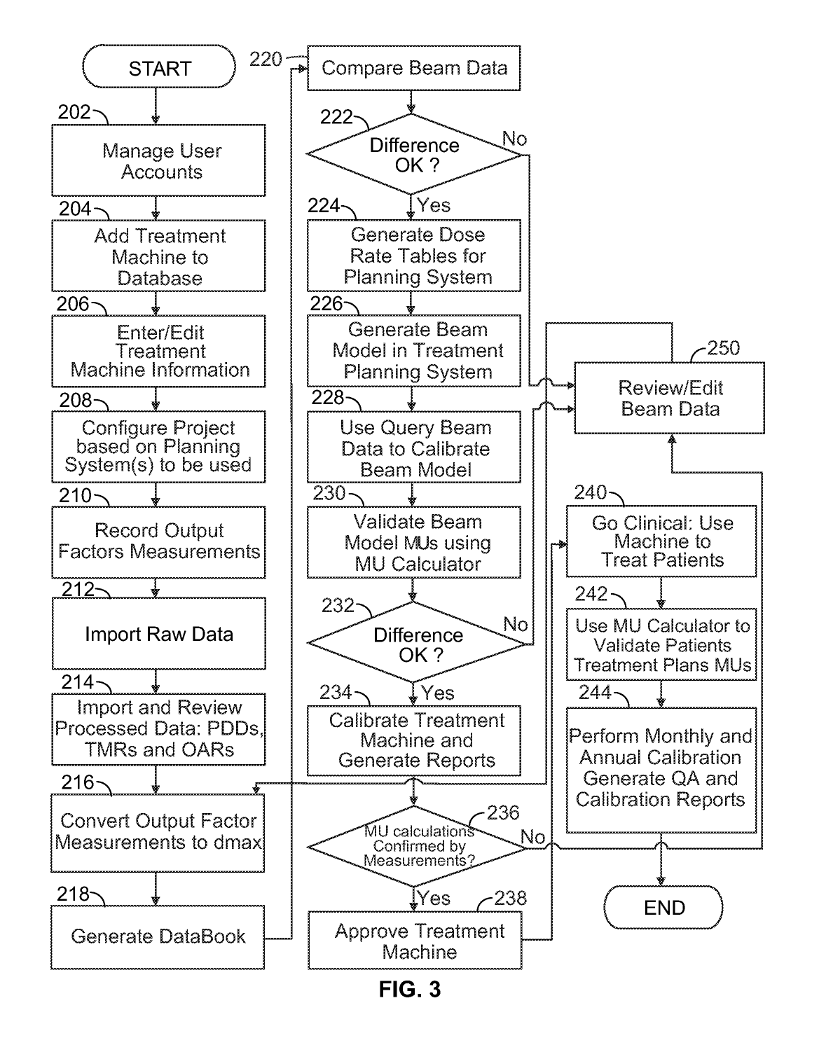

The flowchart in FIG. 3 is an overview of the method for commissioning a RTD according to a preferred embodiment of the invention. In step 202 the RTD to be installed in a clinic is identified and assigned to a designated user(s) by an administrator of the system. There may be more than one user and those users may be one or more of the following: project manager, physicist, clerical staff, other parties. In the preferred embodiment, the assignment is via the assignment module in the system 100. Once assigned, designated users receive a message notifying them of their assignment. Also, throughout the commissioning process, designated users may, in some embodiments, receive notification whenever data is input for the commissioning of the RTD or whenever the input data has been modified.

In the next step 204, a user adds the RTD into the database 106. In this step, the user interface 102 receives identification data for the RTD to be commissioned. The data may include, but is not limited to, data identifying the name of the RTD, its physical location, the type of equipment it is (for example, linear accelerator), a physicist assigned to the RTD and the title of the Data Book to be associated with the RTD. This identification data is stored on the database 106.

For each RTD to be commissioned, the RTD's technical details must be entered into the user interface 102 and stored on the database. In step 206 the machine information for the RTD is entered into the user interface 102 for storage on the database 106. Machine information may include the technical details, the contact information such as the user to be contacted when problems occur with the machine and general information including, but not limited to, historical notes regarding any issues that may have arisen during installation, commissioning and/or servicing of the RTD. This machine information may be entered by the user through the user input 112. If the technical details for a similar RTD are already stored in the database 106, they may be copied and edited as appropriate for the particular RTD now being commissioned. Through the user interface 102, the user may select from a list of previously commissioned RTDs stored on the database and copy the technical detail for one of those other RTDs into data screens for the RTD to be commissioned. If necessary, the data in the various fields may be edited.

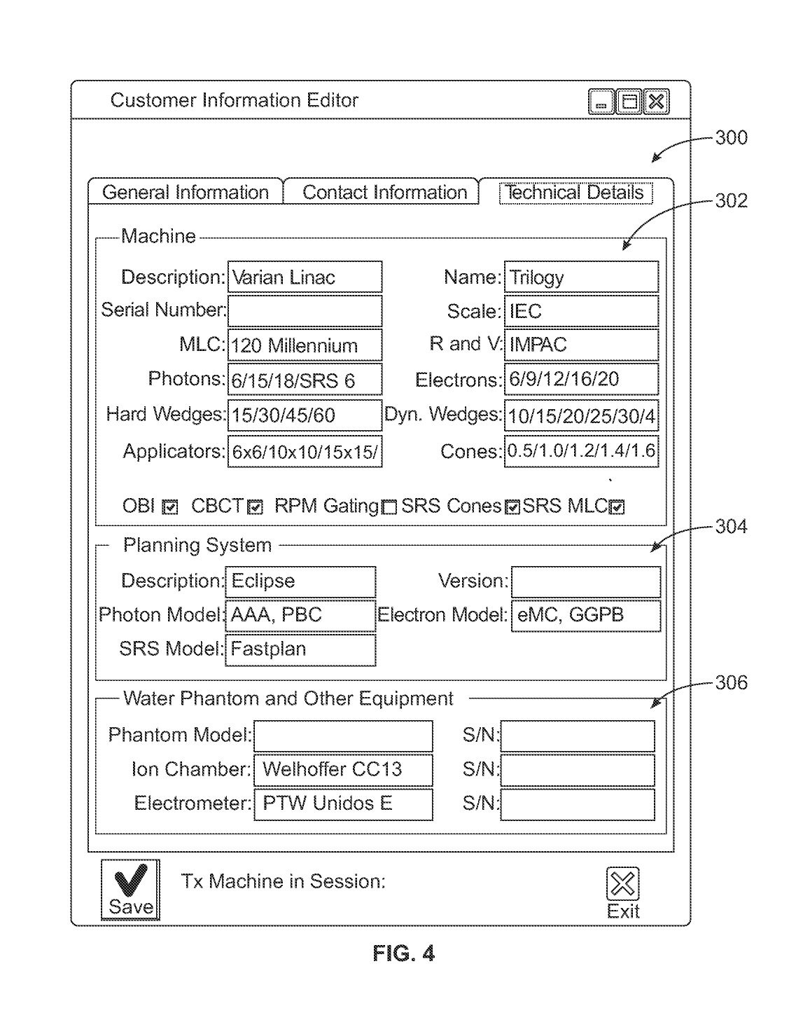

FIG. 4 is in illustration of one embodiment of a screen 300 displaying the technical details entered for a RTD. The screen 300 may include device specific information 302, planning system information 304 relating to the planning system 111 (FIG. 1) to be used with the RTD and test equipment information 306 related to the water phantom 115 or other test equipment such an electrometer 113 or ionization chamber 116 (FIG. 1) used to measure the RTD's output factors. The device specific information 302 may include, but is not limited to, the description and name of the RTD, the serial number, the scale used for the physical position coordinates of the machine, the Multi Leaf Collimator (MLC), the photon energies, the electron energies, and information related to the RTD accessories such as wedges (hard and dynamic), applicators and cones. The hard wedge and the dynamic wedge information identifies the available beam angles. For example, there may be available hard wedges having a 15°, 30°, 45° or 60° angle. Applicator information identifies the field size of the electron beam it defines. For example, a 6×6 applicators identifies an electron applicator defining a 6 cm×6 cm field size at 100 cm SSD (or Source to Surface Distance). Cone information identifies the diameter size of each cone which is the size of the radiation beam, photons in this case, at 100 cm SSD.

The planning system information 304 may include, but is not limited to, the name of the planning system used to validate the beam model, the photon model, the electron model and the Stereotactic Radio Surgery (SRS) model or any other model as required by the planning system. The test equipment information 306 used with the RTD generally may include, but is not limited to, the phantom model and serial number, the ion chamber model and serial number, and the electrometer model and serial number. Other information may be included as well, if desired. Such information is saved to the database 106.

In step 208, Configure Project, the user, utilizing the user interface 102, reviews the default beam configuration parameters and may add other beam configuration parameters for the RTD to be commissioned. The user interface 102 organizes the OF measurements by specific geometry (SSD and depth), beam type (photons, electrons, SRS) and accessory (wedge, applicator or cone). The default set of beam configuration parameters, includes but is not limited to, field sizes, field depths, SSDs and beam accessories for each beam type. Additional parameters that may be added by the user include, but are not limited to, additional field sizes, field depths, SSDs and accessories.

Typically measurements of a RTD's radiation beam are taken in test equipment such as water phantom equipment. Information regarding the strength and depth of the radiation beam at various points is recorded. In step 210, the OF beam measurements are input into the user interface 102. In this step, these OF measurements are those that are not generated by other software associated with the water phantom 115. These OF measurements are measurements, taken by test equipment/measuring devices such as an electrometer and ionization chamber, of the OF for open fields and other accessories that may be used with the RTD. In one embodiment, these OF measurements may be input by the user into the user input 112. In another embodiment, these OF beam measurements may be received by the user interface 102 from the measuring device itself. The measuring device, such as an electrometer 113 (FIG. 1), may be connected to the user interface 102. The user interface 102 detects each electrometer reading and stores it on the database 106. In the preferred embodiment, the user interface detects each measurement reading after the beam being measured is turned off. The measurement reading is then stored on the database 106. The process is repeated until all measurements have been taken.

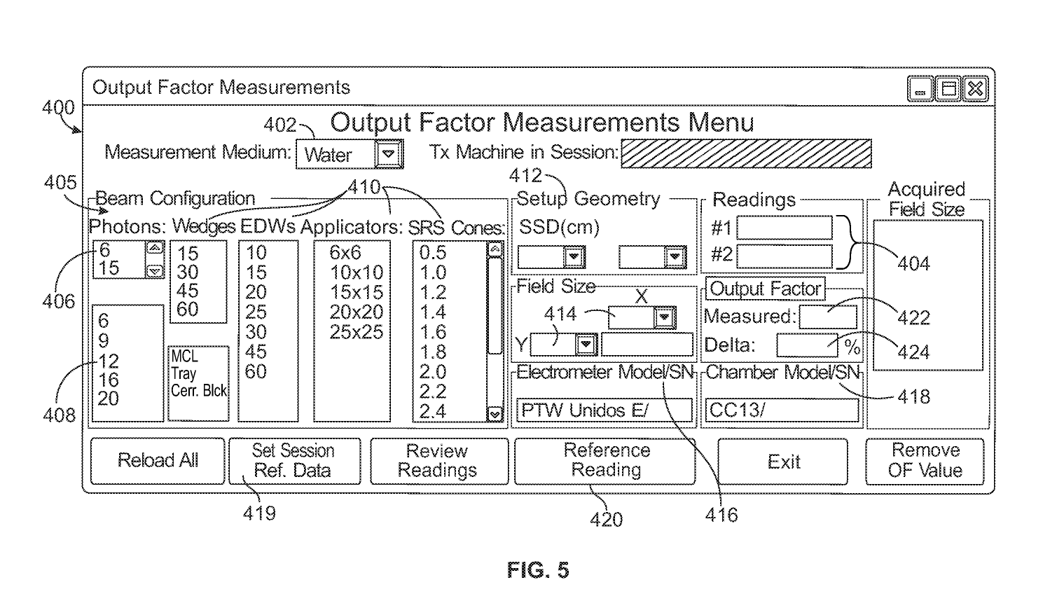

FIG. 5 illustrates one embodiment of a screen, the Output Factor Measurements Menu Screen 400, for entering such OF measurement readings into the user interface 102. In this screen, a user may select the measurement medium 402, typically either water or air, and enter the OF measurement readings 404 for various beam configuration information 405 such as the photon 406 and electron energies 408 for the applicable accessory 410 or open field used with the RTD. Other information that may be selected on the screen 400 may include the setup geometry 412, the field size 414, the electrometer model and serial number 416 and the ion chamber model and serial number 418. For example, to enter measurement readings 404 for the photon energy of 6 with wedge angle 15, the electron energy 408 would be selected and wedge 15 would be selected from the list of wedges. The appropriate setup geometry 412 (in this embodiment SSD and depth) are then selected from drop down menus and the appropriate field size (X and Y coordinates) 414 are selected from drop down menus. The X and Y field size coordinates 414 each appearing in their respective drop down menus are based on the default set of field size parameters and any additional parameters that were added by the user in step 211. The electrometer model and serial number 416 and the chamber model and serial number information 418 are based on the technical details that are saved on the database 106 for the RTD. They can be edited on the user interface 102 if needed. The user enters the measured OF readings 404 into the user interface 102.

The Output Factor Measurements Menu Screen 400 also allows a user to compare the entered OF measurements 404 to an expected value that represents a comparable standard reading for a RTD such as the one being commissioned. Selecting the reference reading button 420 on the screen 400 allows for the reference field size of the beam being commissioned to be measured. The reference field size reading using button 420 may be taken first. Then the actual field size readings may be taken. The ratio of the average of the two set of readings constitute the OF. The resulting ratio of the average readings between the reference field size and actual field size is displayed on the screen as the “Measured OF” 422. The user interface 102 may also calculate and display a delta percent 424 which is the percent difference between the newly acquired Measured OF 422 and the standard reading expected for the RTD. The user may also select other reference data to which OF measurement readings may be compared to determine if they are within acceptable range or not. This other reference data may include data taken from other RTDs. To do this, the user selects the Set Session Ref. Data 419 for a choice of RTDs for which OF measurements are already in the database 106. An RTD may be selected and its comparable data used as a reference for the OF measurements entered on the screen instead of the standard reference measurements built into the system.

In step 212, the raw data from the scan results generated by the test equipment, typically, although not exclusively, water phantom test equipment 115, are imported into the user interface 102 and saved in the database 106 to be later accessed for processing purposes by third party applications or the user interface 102.

Similarly, in step 214, the water phantom scan results that have been processed by third party software are imported into the user interface 102 and saved in the database 106. In the preferred embodiment, these processed results are imported in the form of tables and may include, but are not limited to, open field, wedged field, SRS MLC, SRS cone, and electron PDD tables; open field and SRS cone TMR tables; and OAR tables for open, wedged fields and SRS Cones. The imported tables may be reviewed by the user by displaying the tables on the user interface 102 and may be edited by the user in the user interface 102. These readings are utilized to determine the dose rate per monitor unit (MU) for the RTD. A MU is the unit of beam on-time for the RTD. In the preferred embodiment the MU is calculated by the user interface 102 using the well known Khan's Algorithm.

In step 216, the OF measurements taken at a specific depth are converted by the user interface 102 to the dₘₐₓ values using data in the imported TMR or PDD tables. These dₘₐₓ values represent those at the point of maximum dose. Exemplary calculations are below.

$${OF}_{depth} ( Field Size X ) = \frac{{Reading}_{depth} ( Field Size X )}{{Reading}_{depth} ( Reference Field Size )} = \frac{{OF}_{dmax} ( Field Size X ) \times {TMR}_{depth} ( Field Size X )}{{TMR}_{depth} ( Reference Field Size )}$$

$$Thus$$

$$\begin{matrix}{OF}_{dmax} ( Field Size X ) = {OF}_{depth} ( Field Size X ) \times \\ \frac{{TMR}_{depth} ( Reference Field Size )}{{TMR}_{depth} ( Field Size X )} \\ \approx {OF}_{depth} ( Field Size X ) \times \\ \frac{{PDD}_{depth} ( Reference Field Size )}{{PDD}_{depth} ( Field Size X )}\end{matrix}$$

In step 218 a Data Book is generated. The Data Book may be electronic or hard copy. Measured OFs for open beam are converted to their corresponding values at dₘₐₓ (as above). Wedge Factor OF measurements are converted to Wedge Transmission Factors as follows:

$$Wedge Transmission {Factor}_{depth} ( Field Size X ) = \frac{Wedge {Reading}_{depth} ( Field Size X )}{Open {Reading}_{depth} ( Field Size X )} = \frac{Wedged {Reading}_{depth} ( Field Size X )}{Open {Reading}_{depth} ( Reference Field Size )} \times \frac{Open {Reading}_{depth} ( Reference Field Size )}{Open {Reading}_{depth} ( Field Size X )} = \frac{Wedged {OF}_{depth} ( Field Size X )}{Open {OF}_{depth} ( Field Size X )}$$

OF measurements for different electron applicators taken at different SSDs are converted to Electron OF and Virtual Source to Surface Distances tables. The PDDs, TMRs, OARs Tables included in the Data Book are those imported from the processed scan data (tables) (step 214). All of the above constitute a Data Book which can be left in its electronic format and accessed through the user interface 102 or printed. The Data Book is saved to the database 106.

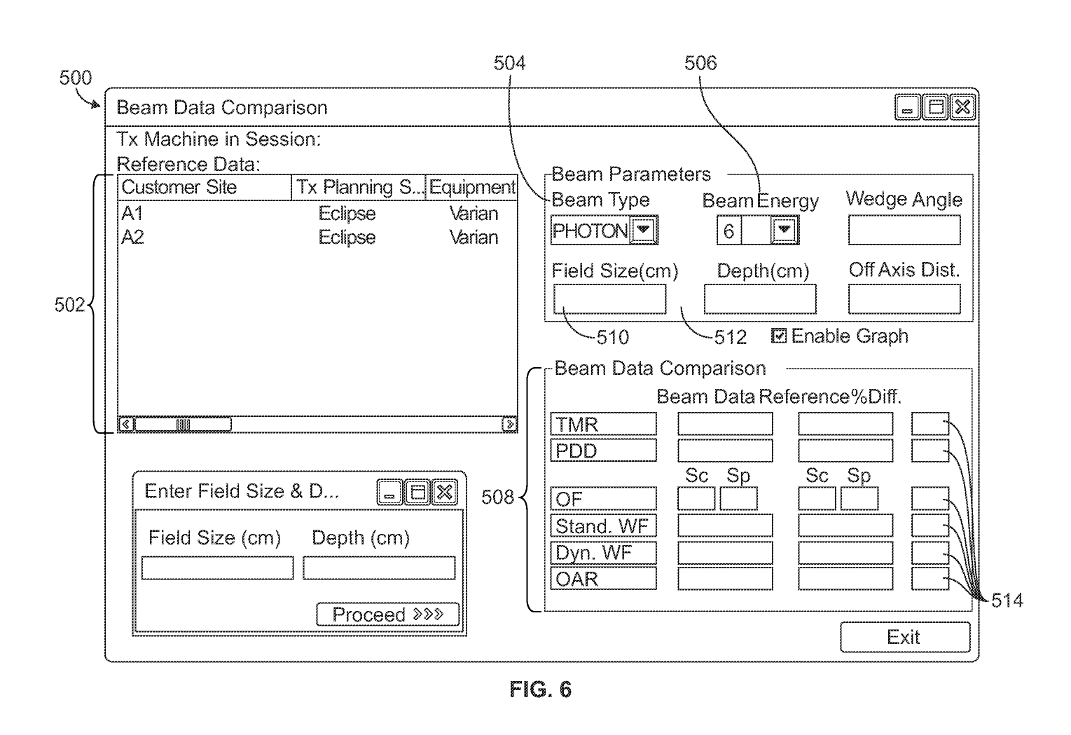

In step 220, the beam data (for example, the TMR, PDD, OF, standard wedge factors, dynamic wedge factors, and OAR) for the uncommissioned RTD may be compared to reference data. The reference data may also be beam data generated by a different RTD and stored in the database 106 or may have been generated by the same RTD at an earlier time period or simply the reference standard treatment machine built into the user interface 102. In some embodiments a summary comparison report of the differences may be created. FIG. 6 illustrates one embodiment of a Beam Data Comparison Screen 500 for comparing newly developed beam data to reference beam data. The user selects the desired reference data from the reference data list 502, selects the beam type 504 and the energy 506 and clicks on the data to be compared 508: TMR, PDD, OF, Standard Wedge Factors, Dynamic Wedge Factors or OAR. The user may make a comparison for all field sizes 510 and depths 512 (when applicable) or for just a specific field size and depth. The percent difference 514 between the newly developed beam data and the reference data is displayed on the user interface 102. A color scheme alerts the user as to the magnitude of percent difference between the two sets of data. Numerical as well as graphical comparisons are displayed on the user interface and may be saved to the database 106.

In step 222, a determination is made as to whether the percent difference between the newly developed beam data and the reference data is acceptable. If so, the user proceeds to step 224 in FIG. 3. If the percent difference does not fall into an acceptable range as recommended for the RTD, then the next step in the process is step 250.

In step 250 the user reviews the beam data for errors, corrects and the process repeats starting at step 216.

In step 224, dose rate tables are generated and input into the treatment planning system. A dose rate table is a table of measured Output Factors that the treatment planning system uses to convert the relative PDD and profile measurements of the beam into absolute dose using the calibration factor of the RTD in the reference beam geometry conditions. Inputs to create the dose rate tables include the OF measurements for open and wedged fields, the desired treatment planning system and its associated algorithm, and the energy and accessory.

In step 226, the user generates a beam model in the treatment planning system 111 (FIG. 1) according to conventional known methods. The beam model is used to generate treatment plans on fictitious patients. The beam model generated by the planning system calculates the radiation dose and dose distributions in the fictitious patient's target tissue and any surrounding organs and structures.

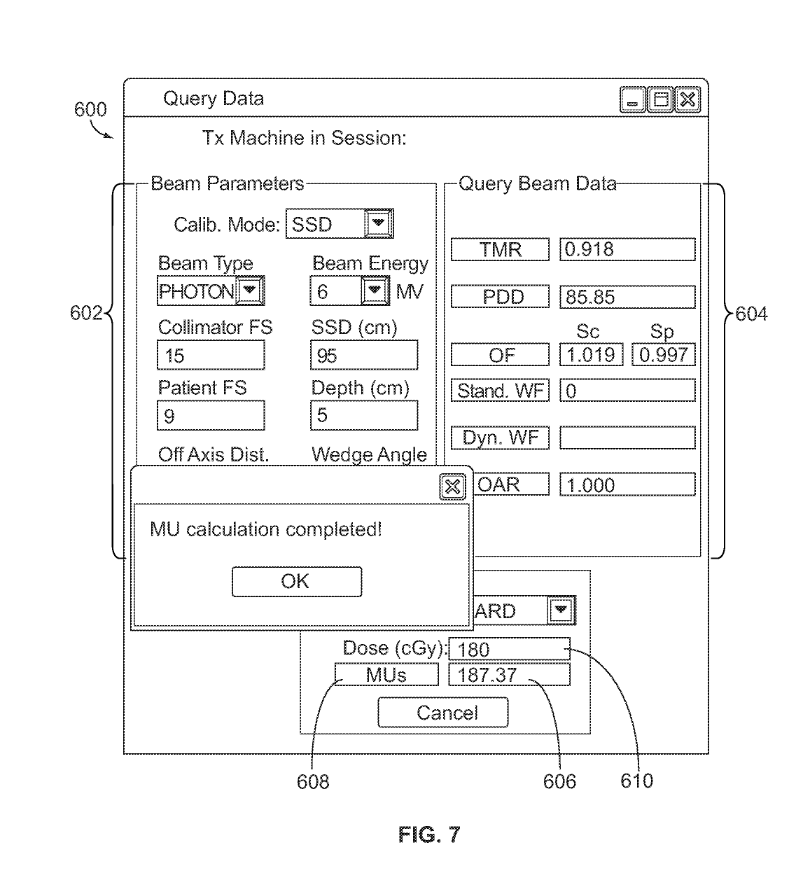

In step 228, the user interface 102 allows for a beam data query with the option of performing a MU calculation for a set of treatment parameters. This is a quick and efficient way for the user to access specific TMRs, PDDs, OFs, Wedge Factors and OARs and during commissioning of a RTD to check for errors in beam data using known reference dose and MUs. FIG. 7 illustrates one embodiment of a Query Data Screen 600 for calculating a MU calculation for a set of treatment parameters. The user may select or input beam parameters 602, including but not limited to, the beam type (photon or electron), the beam energy, the collimator Field Size (FS), the SSD, the patient FS, the depth, the off axis distance, the wedge angle etc. The patient FS is the Field Size to which the patient is actually exposed. For example, the collimator FS may be blocked to shield some critical structure from receiving significant radiation dose. As a result, the field size hitting the patient may be different (smaller) from the one set by the collimator jaws. The beam data 604, are read from the Data Book based on the user selection/entry of beam parameters 602. In addition, the user can enter a dose prescription 606 and activate/click the MU button 608 to calculate the corresponding resultant MUs 610 for the RTD under consideration. In this step the beam models may be calibrated by entering the actual MU required for a given dose in the reference condition of field size, SSD and depth. These calibration values (for example, the number of MUs for a given dose in the reference conditions) are entered into the planning system 111 and are then used by the planning system's beam model to calculate dose and corresponding MUs in any treatment conditions.

In step 230 the beam data is used to perform a preliminary validation (remove gross errors) of the beam model generated by the treatment planning system in step 226. The MUs calculated for each field of the treatment plan by the treatment planning system 111 are verified using the MU calculator in the user interface 102. In other words, comparison is made between the amount of MU derived by the RTD being commissioned and the reference MU calculated by the algorithm used by the planning system 111 using the data from the same RTD. The MU verification requires that the treatment plans from the treatment planning system 111 are exported to the database 106 so that the user interface 102 can calculate its own MUs based on the treatment parameters imported from the planning system 111.

The user interface 102 receives from the planning system the treatment parameters, including the number of MUs, for every treatment field used to generate the plan. As noted above, in step 230, the MU is independently generated using the same treatment parameters from the planning system. These parameters may include, but are not limited to, the beam type, the beam energy, the field size, the beam angle, accessory data, gantry angle, collimator angle, SSD, etc. The treatment field MUs as calculated by the planning system for given treatment parameters should agree within an acceptable range with the RTD's MU calculated and displayed on the user interface 102. In the preferred embodiment the MU and the reference MU from the planning system should be within about 2% difference. In other embodiments, a different range may be acceptable. The percent MU difference may be displayed on the user interface 102. A color scheme alerts the user to the level of discrepancy between the reference MU and the MU for the RTD being commissioned. In the preferred embodiment, a white background means the difference is acceptable, a yellow background means that there may be of cause for concern and a red background signifies that there is a problem to be addressed.

In step 232, the user checks to determine whether the difference between the MU calculated by the RTD and the treatment plan MU is acceptable. If it is not, the next step in the process is step 250. Otherwise, the next step in the method is step 234.

If the difference is acceptable, the user proceeds to calibrate the RTD according to industry standards (step 234). In the preferred embodiment TG-51 is the calibration standard. Calibration entails adjusting the beam output of the treatment machines to yield a given dose in cGy (unit of radiation dose) for a unit MU at a reference depth for a reference field size at either 100 cm Source to Surface Distance (SSD) or Source to Axis Distance (SAD). In step 234, all calibration parameters are saved and accessed as needed for future use. The list of calibration reports are generated and stored by date in the database 106 for later review.

In step 236, a comparison is made between the dose for a set of MUs calculated by the treatment planning system and that measured at the machine for the same number of MUs in the conditions set by the treatment plan. If agreement is satisfactory, the treatment machine is approved (step 238). If not, the user goes back to step 250 to review and edit the treatment machine data.

In step 240, once the commissioning is complete, the RTD is ready to be used in a clinical setting to treat patients.

In step 242 the user interface 102 serves the clinic on a daily basis through verification of patient's treatment plans (MU Calculations), RTD quality assurance and associated report generation, and monthly calibrations. For example, in clinical settings where the patient must be treated, in the absence of a prescribed treatment plan by the treating physician, the beam data query functionality on the Query Data Screen 600 (FIG. 7) of the user interface 102 may be used by appropriate clinical staff member to perform a MU calculation for a set of treatment parameters found in the patient's medical charts. This Query Data Screen 600 (FIG. 7) allows the user to query beam parameters, such as a PDD, TMR, OAR, OF, Transmission Factor, etc, now residing in the Data Book for the RTD. It also allows a treating staff member to enter set up conditions and quickly calculate the required MUs. This is very useful in practice since some patients needs emergency treatment and there is no time to perform a full treatment plan on them. The user interface 102 may generate a report, as required clinically, for documentation purposes based on entered and generated data including the name of the patient, the diseased target to be treatment, the identification of the treatment field, the treating RTD etc.

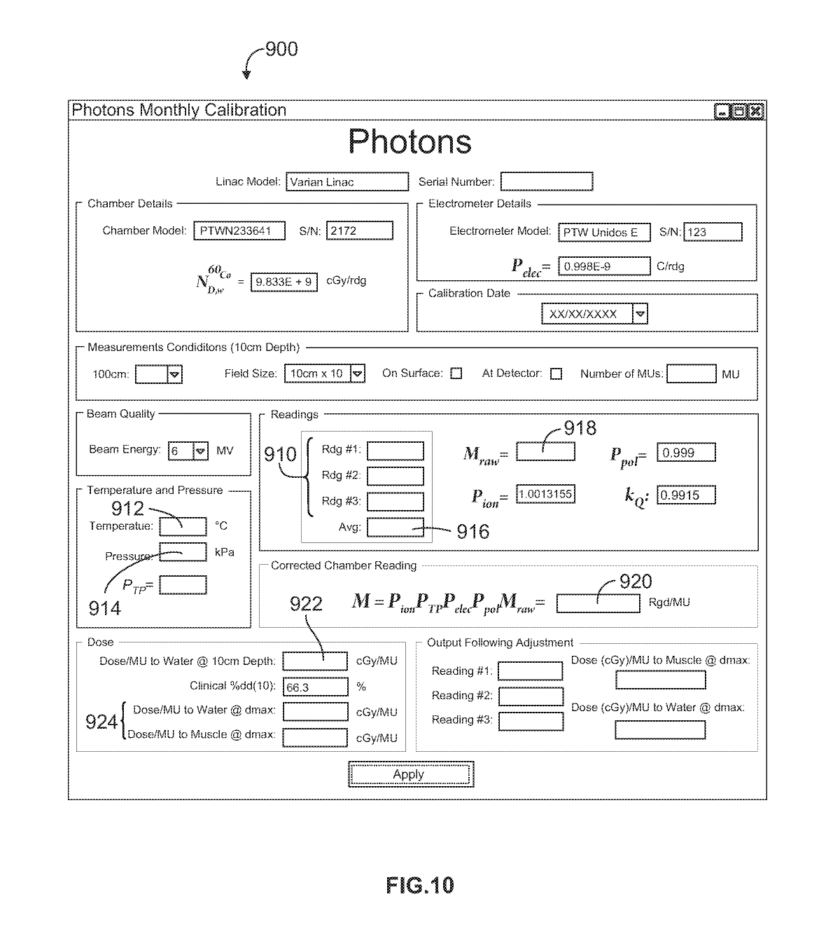

In step 244 monthly calibration of the RTD is performed. Calibration parameters are often needed during monthly calibration where, by regulation, the output of the RTD is checked to be within a specified percentage, in mostly within 2%, of the 1 cGy/MU. If not with the specified percentage, the output of the RTD is adjusted. During the monthly calibration, no new calibration parameters are generated but a monthly report may be electronically generated. To perform a monthly calibration, a user sets the RTD to be calibrated to a desired number of MUs (beam on time). The user enters the beam type and energy to be calibrated into the user interface 102. The Monthly Calibration Screen 900 illustrated in FIG. 10 is used to enter new ion chamber readings (readings 1-3) 910 in reference condition. The user enters the temperature 912 and pressure 914. The user interface calculates the average reading 916, the M_{raw} value 918, Corrected Chamber Reading M 920, Dose/MU at 10 cm depth 922 (measurement point in water) and dₘₐₓ 924 per calculations known to those of skill in the art. In the preferred embodiment, if a difference of more than 2% is found, adjustment to the beam output is performed to yield 1 cGy/MU at dₘₐₓ either for water or muscle. After calibration, the user interface may generate a corresponding report of the calibration.

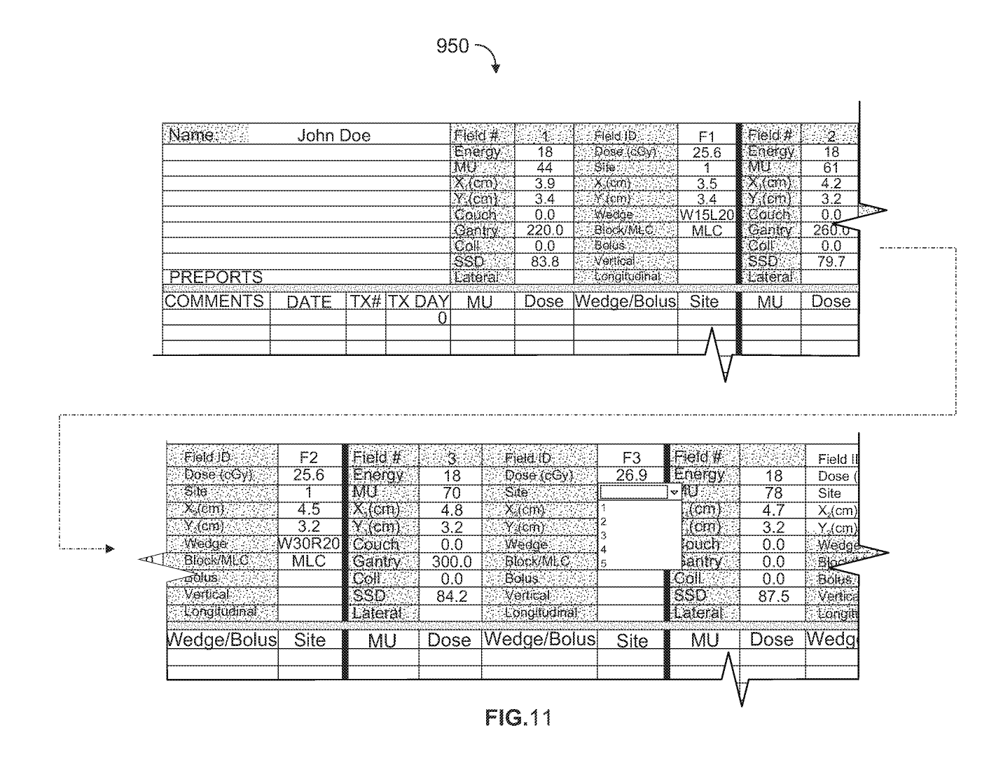

Because of all of the data for the RTD that is stored in the database 106, the user interface 102 provides a virtual RTD treatment machine accessible through its user interface 102. As a result, technologies developed for use in conjunction with the RTD can be managed through the user interface 102. One such example is Electronic Medical Records of the patients scheduled to be treated on a particular RTD. The user interface 102 allows a staff member to click on a menu item called “Patients On Treatment.” This opens a list of patients scheduled to be treated on a particular day. The user may click on a patient's name to open his/her electronic chart 950, as illustrated in FIG. 11, where details about the MUs delivered in the past to the patient have been entered by the appropriate staff members. Through the user interface, comments may be added to the chart. Before closing the chart, an electronic signature of the treating staff may be added for documentation purposes. These charts or e-charts may be accessed by the treating physician or physicist for review using the user interface 102. They also serve as a quality assurance tool for the treatment records of the machine which are know to have issues as far as loss of communication to and from the machine itself or to have corruption in their database.

The system 100 also has a document manager module to organize the documentation associated with the RTD. Documents may include those associated with the purchase, acceptance testing, commissioning or any other aspect of the RTD throughout the lifetime of the RTD.

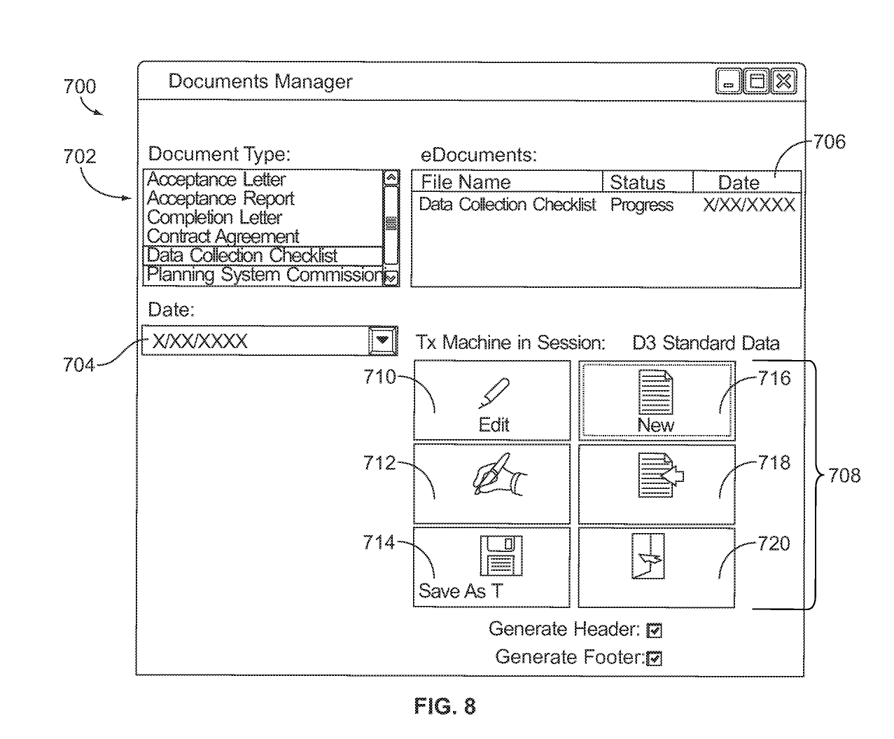

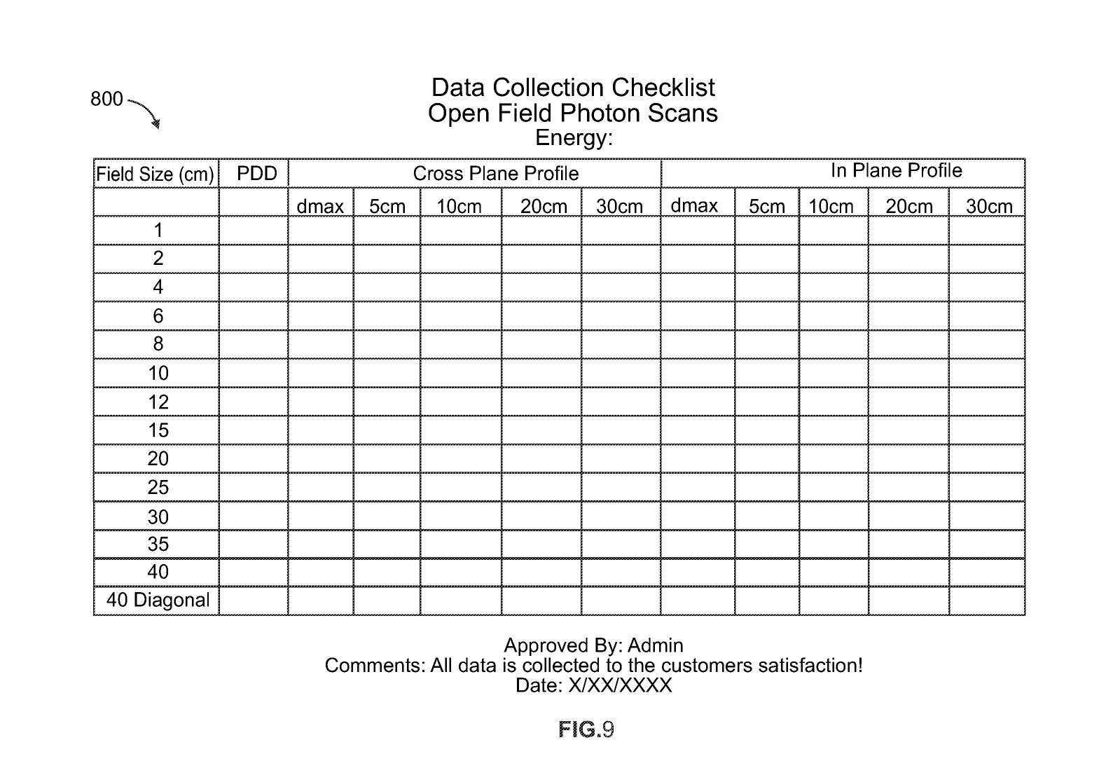

FIG. 8 illustrates one embodiment of a Machine Related Documents screen 700. This screen may include a document type area 702, a date area 704, a status area 706 and function buttons 708. The function buttons may include, but are not limited to, the following: edit 710, approve 712, save as template 714, new 716, import 718 and exit 720. In the screen illustrated in FIG. 8, initially no documents will be present for a given RTD. Tools are provided to help create, import, edit, save as templates and electronically approve documents. A list of documents types may be given in the document type area 702 on the screen. Document types may include, but are not limited to acceptance letters, acceptance reports, completion letters, contracts and the Data Collection Checklists. For example, to create a Data Collection Checklist, a user may select “Data Collection Checklist” from the list in the document type list area 702 on the screen 700 and then select the creation date 704 and the function button “New” 716. A blank Data Collection Checklist 800, such as the one embodied in FIG. 9, will be generated, added to the eDocuments list displayed in the status area 706 and assigned a progress status. The user may fill out the document via the user interface 102 and save the document to the database 106.

In another example of a document created by the document manager module is a commissioning report. To create such a report, the machine technical details, the results of TG-51 calibration, the beam data residing electronically in the Data Book in the database 106 are fed by the user interface into the commissioning report template. Similar templates are available through the user interface 102 for other reports that use RTD information entered on the user interface 102 or stored in the database 106.

In the preferred embodiment there may be documents created using the user interface 102 or stored on the database 106 that need to be approved by one or more users. Some documents may be electronically signed by one authorized user and other types of documents may require approval by a plurality of users.

In an embodiment, a document is generated. It may be edited as necessary by the user on the user interface 102. A qualified user (for example, qualified medical physicist authorized in the user interface to approve documents) may review the document and if satisfied, close the document or, alternatively, make changes and save and close the document. To approved or sign the document, the authorized user clicks an “Approve” button 712. A new window appears where comments can be entered as part of the approval process. When an “Apply” button is pressed, a note is added to the footer or header of the word document indicating the user who approved the document, the corresponding approval note/comment and the date of approval. The document, at this stage, is also password protected and may not be edited unless it is a document that requires two signatures. In the scenario where it is a document that requires two signatures, the document will only be locked for further editing when the second signature is added.

For example, the Data Collection Checklists may need the approval of both a commissioning physicist and another party. In this exemplary embodiment, the status of approved documents is changed from “Progress” to “Approved” except for the Data Collection Checklist which moves from “Progress” to “PartialA” (partially approved) to “Approved” (when the second eSignature is applied). Once a document is approved, it cannot be edited or modified. In one embodiment, electronic signatures may be placed at the footer of the document when only one eSignature is required and may appear at both the header and footer when two signatures are needed for approval.

Another exemplary document that may be created and approved using the user interface 102 is the TG-51 or Monthly Calibration Report. The report may be generated by the user through the user interface 102, edited on the user interface and saved to the database 106. The report may be approved by an authorized user by clicking on the “Approve” button 712 (FIG. 8). Once approved, the document is locked from further editing.

The user interface 102 also allows a user to import the data from another machine into the database 106. Similarly, the user interface also allows the data associated with another machine to be exported.

The system or systems may be implemented on any form of computer or computers and the components may be implemented as dedicated applications or in client-server architectures, including a web-based architecture, and can include functional programs, codes, and code segments. Any of the computers may comprise a processor, a memory for storing program data and executing it, a permanent storage such as a disk drive, a communications port for handling communications with external devices, and user interface devices, including a display, keyboard, mouse, etc. When software modules are involved, these software modules may be stored as program instructions or computer readable codes executable on the processor on a computer-readable media such as read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion. This media can be read by the computer, stored in the memory, and executed by the processor.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as Visual Basic, Visual Basic.Net, C, C++, Java, assembler, or the like, with the various algorithms being implemented with any combination of data structures, objects, processes, routines or other programming elements. Furthermore, the present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. A method for commissioning a radiation treatment device (RTD), comprising:
entering identification data for an uncommissioned RTD into an RTD database;
entering machine information for the RTD into the RTD database;
importing into the RTD database processed scan data generated by scanning equipment measuring beam characteristics of the RTD's beam;
entering additional data from measurement results generated by test equipment measuring aspects of the RTD's beam into the RTD database;
creating a Data Book in the RTD database comprising output factors, transmission factors and processed scan data;
comparing information in the Data Book with information according to a predefined standard for commissioning the RTD;
comparing a treatment plan MU based on a set of treatment parameters to the RTD MU calculated using the set of treatment parameters; and
commissioning the RTD based at least in part on if the comparing meets the predefined standard and if the MU calculated by the RTD is within an acceptable range of the treatment plan MU.

2. The method of claim 1, wherein the identification data comprises a location of the RTD and a name of a physicist assigned to the RTD.

3. The method of claim 2, wherein the technical details for the RTD are copied from a different RTD.

4. The method of claim 2, wherein the machine information comprises technical details for the RTD, the technical details including device information, planning system information and the equipment information.

5. The method of claim 4, wherein the device information includes the photon energies and the electron energies for the RTD.

6. The method of claim 4, wherein the test equipment information comprises a water phantom model and serial number.

7. The method of claim 1, wherein the processed scan data comprises open field, wedged field, SRS MLC, SRS cone and electron PDD tables.

8. The method of claim 1 further comprising displaying the processed scan data on a user interface and editing at least a portion of the processed scan data.

9. The method of claim 1, wherein the additional data are output factor measurements for open fields and the test equipment measuring the output factor measurements is an electrometer.

10. The method of claim 9, wherein the additional data entered is electronically transferred from the electrometer to the RTD database.

11. The method of claim 9 further comprising comparing at least one of the output factor measurements to a reference value.

12. The method of claim 1, wherein the predefined standard is based on beam data generated by a different RTD and stored in the RTD database.

13. The method of claim 1, wherein the set of treatment parameters comprise beam type, beam energy and field size.

14. The method of claim 1 further comprising using, after the commissioning of the RTD, the RTD to calculate MUs for a patient based on treatment parameters input into the user interface.

15. A system for commissioning a radiation treatment device (RTD) comprising:
a server including a processor and a data store including an RTD database, the server accessed by the user interface, the server retrieving data related to the commissioning of the RTD;
an user interface comprising an input and an output;
first test equipment that has an output providing processed scan data obtained from the RTD;
second test equipment that has an output providing additional data from the RTD;
a first algorithm deriving a Data Book residing in the RTD database comprising output factors, transmission factors and processed scan data;
data representing a predefined standard for commissioning the RTD;
a second algorithm for comparing information in the Data Book with information according to a predefined standard for commissioning the RTD; and
a third algorithm for comparing a treatment plan MU based on a set of treatment parameters to the RTD MU calculated using the set of treatment parameters.

16. The system of claim 15, wherein the first test equipment is a water phantom and the second test equipment is an electrometer.

17. The system of claim 15, wherein the status of a document associated with the RTD is retrieved by the server from the RTD database and displayed on the user interface.

18. The system of claim 17, wherein the document is a data collection checklist and at least one approval is displayed on the document.

19. The system of claim 15, wherein a planning system provides the third algorithm with the treatment parameters.

20. The system of claim 14, wherein the additional data comprises output factor measurements and at least a portion of the output factor measurements entered into the user interface for field size, field depth and SSD are for beam configuration parameters added by a user during configuration of the project.

21. A computer program product, comprising a computer usable medium having a computer readable program code embodied therein, the computer readable program code adapted to be executed to implement a method for commissioning a radiation treatment device (RTD), the method comprising:
entering identification data for an uncommissioned RTD into an RTD database;
entering machine information for the RTD into the RTD database;
importing into the RTD database processed scan data generated by scanning equipment measuring beam characteristics of the RTD's beam;
entering additional data from measurement results generated by test equipment measuring aspects of the RTD's beam into the RTD database;
creating a Data Book in the RTD database comprising output factors, transmission factors and processed scan data;
comparing information in the Data Book with information according to a predefined standard for commissioning the RTD;
comparing a treatment plan MU based on a set of treatment parameters to the RTD MU calculated using the set of treatment parameters; and
commissioning the RTD based at least in part on if the comparing meets the predefined standard and if the MU calculated by the RTD is within an acceptable range of the treatment plan MU.
